(19) United States
(12) Patent Application Publication
(10) Pub. No.: US 2026/0053595 A1
(43) Pub. Date: Feb. 26, 2026

(54) ENTRY POINT IDENTIFICATION SYSTEM

(71) Applicant: MARROW WIZ LTD., Rehovot (IL)

(72) Inventors: Liraz SHLOMOFF, Rehovot (IL); Yakov NEDLIN, Hermesh (IL); Matan Yakov ELBAZ, Rehovot (IL); Ofer VIKINSKY, Tzur Yigael (IL)

(21) Appl. No.: 19/104,653

(22) PCT Filed: Sep. 12, 2023
(86) PCT No.: PCT/IL2023/050986

§ 371 Date: Feb. 18, 2025

Related U.S. Application Data
(60) Provisional application No. 63/406,105, filed on Sep. 13, 2022.

(51) Int. Cl. A61B 90/98; A61B 17/34; A61B 34/20

(52) U.S. Cl. CPC A61B 90/98; A61B 17/3403; A61B 34/20

(57) ABSTRACT

The present invention pertains to an entry point location system. The system features a sensor device, which includes a plurality of spaced apart sensors for external reversible attachment to a patient at a target site. The sensors are configured to transmit sensed data. The system features a computing device. The sensors are in communication with the computing device and the computing device is configured to receive the data from the sensors and use the data to calculate the location of an entry point. The system may include a guiding tool for guiding a user to access an entry point. In addition, the present invention is of a method of determine the location of an entry point.

### FIELD OF THE INVENTION

The present invention relates to an entry point identification system. Moreover, the present invention is of a system for identifying and guiding a user to an optimal point for inserting a needle in a bone.

### BACKGROUND OF THE INVENTION

In bone marrow aspiration and bone marrow biopsy a needle is usually inserted into the bone and a substance is withdrawn. Bone marrow aspiration is typically done without the aid of imaging techniques such as X-ray, ultrasound and computed tomography (CT). Typically, the doctor identifies where to insert the needle using touch and sight. The doctor locates anatomical landmarks and navigates from that point. For example, in bone marrow aspiration in the tibia, the doctor may locate the joint line and then may navigate a suitable distance distally along the medial plane and medially. This method is not very accurate and can be further hindered by the physical condition of the patient at the target area, such as for example swelling and obesity. It is critical to identify the landmarks precisely for correct and optimal bone marrow aspiration. If the point of entry is not correct, the procedure may cause damage to the patient and may not provide the bone marrow, or the amount of bone marrow needed.

It would be desirable to have a system for accurately locating a point of entry for an invasive procedure, such as in a bone. It would be advantageous if the system would provide reproducible results and would be facile to use. It would be beneficial to have a reliable method for locating a point of entry in a bone to be used in bone aspiration, which does not depend on human estimation. The present invention provides such a system and method of use thereof.

### SUMMARY

The invention may have several aspects. One aspect is of an entry point location system. The system may feature a sensor device and a computing device. The sensor device may include a plurality of spaced apart sensors for external reversible attachment to a patient at a target site, wherein the sensors are configured to transmit sensed data. The sensors are in communication with the computing device and the computing device is configured to receive the data from the sensors and use the data to calculate the location of an entry point.

In various embodiments of the system, each of the plurality of sensors may include a load cell for measuring load or displacement. Each of the plurality of sensors may include a bridge resistor for measuring strain. The plurality of spaced apart sensors may be disposed longitudinally on the skin facing side of a patch. The patch may include an adhesive on the skin facing side of the patch for detachable attachment to the skin of a patient. The patch may be sized and shaped to be placed on the knee, such that the plurality of spaced apart sensors are disposed between about the patella to below the tibial tuberosity. The computing device may be at least one of a computer, a microcomputer, a smartphone, a tablet and a smartwatch. The plurality of sensors may be coupled by wires to the computing device. The sensor device may include an attachment component for coupling to the computing device for facilitating communication from the plurality of sensors to the computing device. The computing device may power the plurality of spaced apart sensors. The plurality of sensors may be coupled wirelessly to the computing device. The sensor device may include at least one antenna and at least one power source. Each sensor of the plurality of sensors may include an identification means corresponding to the sensor to facilitate a user identifying the sensor. The identification means may be disposed on the external facing side of the sensor device. The identification means may be a led. The identification means may be an identification number. The system may be configured to collect data from the plurality of sensors when the target site is flexed and when the target site is extended. The target site may be a knee and wherein the sensor device may be configured to attach to the knee for attachment of the plurality of sensors on the midline between the patella and the tibial tuberosity along the anterior crest of the tibia and wherein the sensor device includes a section spaced apart from the plurality of sensors, the section featuring a plurality of openings positioned on the horizontal lines of each of the plurality of sensors, for providing an opening for needle insertion at the determined entry point. The entry point may correspond to a point on the radial line defined by a sensor which senses a strain or a load and wherein the computing device calculates a difference between two bending states of the target area at the sensor of zero.

In various embodiments of the location system, the system may further include a guiding tool for guiding a user to an entry point. The guiding tool may feature a section of the sensor device, the section spaced apart from the plurality of sensors, the section including a plurality of openings positioned on the horizontal lines of each of the plurality of sensors, for providing an opening for needle insertion at the determined entry point. The guiding tool may include a base substrate and attachment means. The base substrate may include an alignment means and an access hole. The alignment means may be for aligning the tool with the sensor determined to be positioned at an entry point. The access hole may be spaced apart and in the same horizontal line as the alignment means, the access hole configured for providing access to an instrument to an internal target site. The attachment means may be for attaching the guiding tool to the sensor device. The alignment means may include an opening configured to overlie a marked position of a sensor on the external side of the sensor device.

A further aspect is a method of determining the location of an entry point. The method may include attaching the sensor device to a target area of a patient. The method may include sensing data by the at least one sensors. The method may include transmitting the data to the computing device. The method may include calculating the strain. The method may include indicating to the user the sensor for which a difference in the strain in two bending configurations of a target area is measured by the computing device as approximating to zero. The method may include determining the entry point about the sensor corresponding to the difference in strain of zero. The method may include extending the target area of the patient before the sensing data. The method may include flexing the target area ninety degrees before the sensing data. The target area may be the knee.

In various embodiments of the method of determining the location of an entry point, attaching the device may features attaching with an adhesive to facilitate contact of the plurality of spaced apart sensors with the skin of the patient. The device may include a patch and wherein the attaching the device may include attaching the patch to the patient. The attaching the device may feature connecting the device to the computing device. The attaching the device may feature the computing device providing power to the sensors. The indicating to the user may include illuminating a led coupled to the sensor. The determining the entry point may include finding an entry point on the radial line defined at the position of the sensor indicated to the user. The finding an entry point on the radial line may include attaching the guiding tool, so that the opening of the alignment means overlies the marked position on the external side of the sensor device of the sensor corresponding to a difference in strain of zero to provide the user with an entry point at the spaced apart access hole. The sensor device may feature a section spaced apart from the plurality of sensors, the section including a plurality of openings positioned on the horizontal lines of each of the plurality of sensors, for providing an opening for needle insertion at the determined entry point and wherein finding an entry point may include finding the opening corresponding to the sensor indicated to the user for insertion of a needle in the opening.

### BRIEF DESCRIPTION OF THE DRAWINGS

The various features of the invention will best be appreciated by simultaneous reference to the description which follows and the accompanying drawings, which are not drawn to scale and in which:

FIGS. 1a and 1b show a front view (FIG. 1a) and a perspective view (FIG. 1b) of an exemplary entry point location system according to an aspect of the present invention;

FIG. 2 shows an exemplary sensor device according to an aspect of the present invention;

FIG. 3 shows a perspective view of an exemplary computing device according to an aspect of the present invention;

FIGS. 4a and 4b show respectively an exemplary sensor device coupled to the knee wherein the knee is in an extended configuration (FIG. 4a) and a bent configuration (FIG. 4b) according to an aspect of the present invention;

FIG. 5 shows a perspective view of a portion of an exemplary electrical connection of an exemplary sensor device according to an aspect of the present invention;

FIGS. 6a and 6b show an exemplary sensor device coupled to an exemplary computing device according to an aspect of the present invention;

FIG. 7 shows an exemplary wireless entry point location system according to an aspect of the present invention;

FIGS. 8a, 8b, 8c and 8d show various views of an exemplary user alert according to an aspect of the present invention;

FIGS. 9a and 9b show front views of an exemplary entry point location system with an exemplary user alert according to an aspect of the present invention;

FIGS. 10a-10g show exemplary guiding tools for use with an exemplary sensor device according to an aspect of the present invention;

FIG. 11 shows a view of an exemplary sensor device according to an aspect of the present invention; and

FIG. 12 shows a flow chart of an exemplary method of using an exemplary entry point location system according to an aspect of the present invention.

### DETAILED DESCRIPTION

In one aspect the present invention is of a system for identifying an entry point for an invasive procedure, such as, but not limited to bone aspiration. In a further aspect, the present invention provides a method of using the entry point identification system of the present invention to determine the location of an entry point in a target site.

The system and methods of use thereof of the present invention have many advantages. The system provides reliable identification of an optimal entry point for an invasive procedure. The system may be used by different users and the results may be reproducible with each user. The system may reduce the error associated with the human estimation inherent in the methods of the art. In addition, the accuracy of the system and method of use thereof may provide a better outcome from the invasive procedure. For example, in bone marrow aspiration, the precision of the point of entry may facilitate a greater collection of bone marrow.

The term ‘consisting essentially of’ as used herein means that the scope is limited to the specified elements and those that do not materially affect the basic and novel characteristic(s) of the claimed device and materials.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

All ranges disclosed herein include the endpoints. The use of the term “or” shall be construed to mean “and/or” unless the specific context indicates otherwise.

The term ‘about’, in some embodiments, refers to ±30 % of the stated numerical value. In further embodiments, the term refers to ±20 % of the stated numerical value. In yet further embodiments, the term refers to ±10 % of the stated numerical value.

As used herein the term ‘optimal point of entry’ may refer to any suitable point along a portion of an identified radial line of a target area of the body. The identified radial line may correspond to the position on the skin of a sensor identified by a computing device as located at a point of entry. The term encompasses the most suitable point of entry identified by the herein invention.

As used herein the terms ‘a’ and ‘an’ may mean ‘one’ or ‘more than one’.

As used herein the terms ‘comprising’, ‘including’, ‘containing’, ‘featuring’, ‘having’ and any forms of the terms thereof are inclusive and open ended and do not exclude additional elements or method steps, which are not recited.

The principles and operation of a system configured to determine a point of entry in an invasive procedure, as well as methods of use thereof according to the present invention may be better understood with reference to the figures. The figures show non-limiting aspects of the present invention.

The present invention provides an invasive procedure entry point identification system. The system may be used for locating an entry point for any suitable invasive procedure, such as, but not limited to bone marrow aspiration. A target site may feature a plurality of entry points along a defined section of the radial line corresponding to an identified entry point at the target site. The system may include a plurality of sensors and a computing device. The system may further include a guiding device.

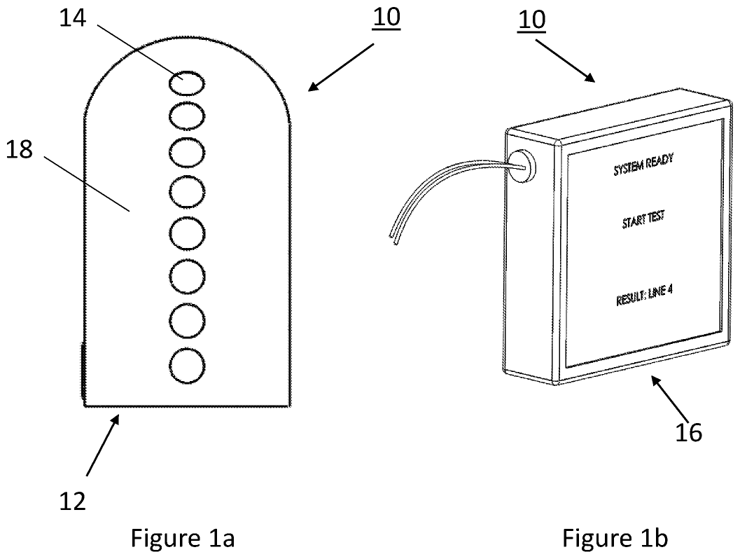

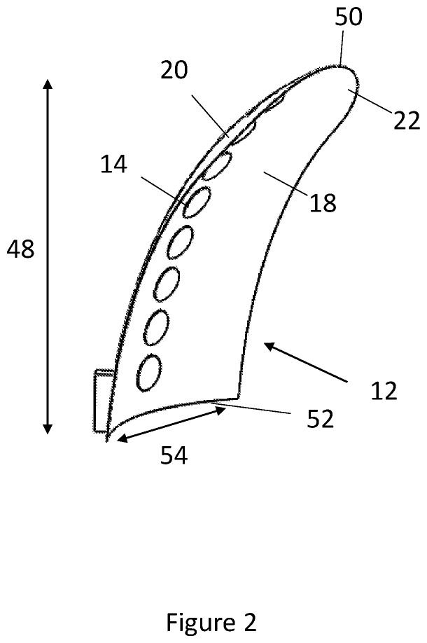

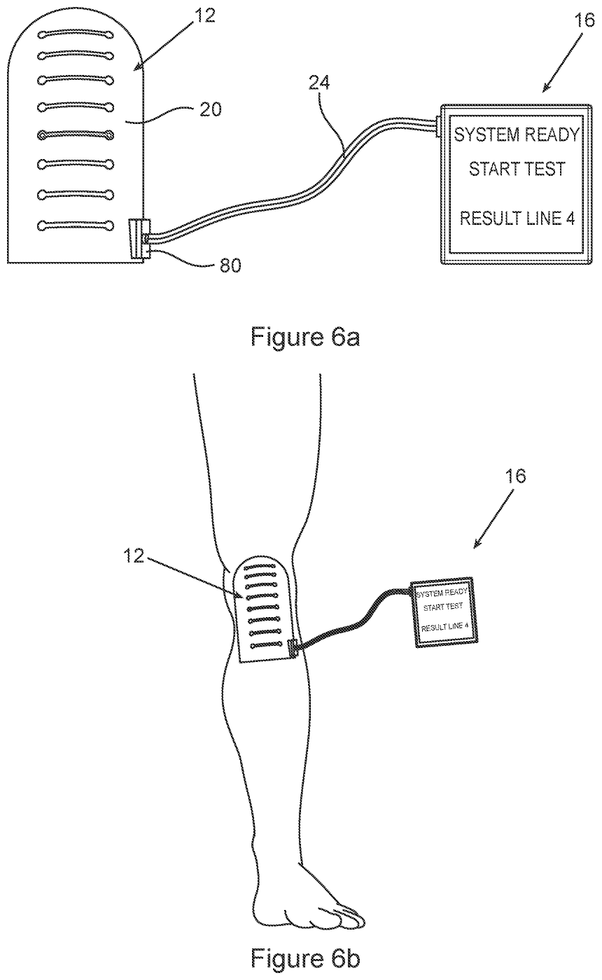

FIGS. 1a and 1b show an exemplary entry point identification system 10 according to an aspect of the present invention. System 10 may include a sensor device 12 featuring a plurality of sensors 14 and a computing device 16. The sensor device 12 may be flexible and have a skin facing side 18 (FIG. 1a) and an external facing side 20 (see for example FIG. 6a). FIG. 2 shows the inner side 18, configured to be skin facing, of an exemplary sensor device 12. The sensor device 12 may include a flexible base substrate 22. The sensor device 12 may be configured as a patch. As used herein the term “sensor device” is interchangeable with the term “patch”. Patch 12 may be constructed from any suitable material for application on the skin. Patch 12 may include an attachment component (not shown in the Figures) for attaching to a target area of the body. One non-limiting example of an attachment component, which is also conducive for ready detachment of the patch by a user is an adhesive (not shown). The layer of adhesive may include a protective layer to cover the adhesive before application of the sensor device, such as a peelable liner. Other non-limiting examples of attachment components include elastic straps, ties, clips, and braces, which may be suitably coupled to the patch 12 and which may secure the patch to the target area of the body. A patch 12 with an elastic attachment component may feature an elastic bandage. The inner face 18 of the patch 12 may be the skin contacting side of the device. The patch 12 may be sized and shaped for attachment to a target area. Patch 12 may be sized and shaped for accurate placement at a target site. In one non-limiting example the target site is the knee. The patch 12 may be shaped so that it can be coupled to the knee and to suitable distanced adjacent areas for locating an entry point within the patch covered site. The patch may be made from a flexible material that can adapt the shape of the knee. In one non-limiting example of a sensor device 12 for use on the knee, the patch may be configured so that when applied to the knee, at least a section 48 of the patch featuring a plurality of sensors 14 disposed on the patch are attached spaced apart on the midline between the patella and the tibial tuberosity along the anterior crest of the tibia. Patch 12 may have dimensions of from about 150 mm to about 250 mm in length, and from about 30 mm to about 100 mm in width.

The sensor device 12 may include any suitable number of sensors 14 disposed on the skin facing side 18 of the patch. In some embodiments, device 12 may include an array of at least five sensors 14. For example, device 12 may include at least 6 sensors, at least 7 sensors, at least 8 sensors, at least 9 sensors, or at least 10 sensors. Each possibility represents a separate embodiment of the invention. In some embodiments, device 12 may include an array of up to about fifteen sensors 14. The sensors 14 may be spaced apart at a suitable distance from each other. The less distance between the sensors 14 the more accurate the determination of the entry point may be as more regions of the target body area are being monitored. The sensors 14 may be spaced apart longitudinally 48 extending between the proximal end 50 and the distal end 52 of the device 12. In an embodiment (not shown in the figures), more than one sensor 14 may be positioned along the width 54 of the patch 12. Sensors 14 may be independent from each other and may not be coupled together. Sensors 14 may be any suitable sensor for measuring a suitable property of the target area to which it is coupled, such as but not limited to the ligaments, tendons, muscles and bone of a target body area. Sensors 14 may be for monitoring and measuring at least one of the load, strain, deformation, displacement and stretching of a target body area. The sensors 14 may be configured to measure deformation resulting from flexion of a target area. Non limiting examples of a suitable sensor 14 may include at least one of a bridge resistor, a strain gauge, a Wheatstone bridge and a load cell. The sensors 14 may be configured to measure the property of the target area in any position or state of the target area, such as when it is bent or straight. For improved results the difference between these two states may be measured. In an example wherein the target area is the tibia, the leg may be outstretched, and the leg may be bent at the knee. An optimal point of entry may be a point on the bone wherein there is no measured strain or stretching difference between full extension and bending. There may be more than one point representing no difference in strain or tension, but the algorithm of choosing the optimal point should pick up the most upper one, which represents the tibial tuberosity. Different algorithms should be evaluated and defined for different applications and anatomies. The sensor device 12 may be for one time use or may be for multiple reuses. The sensor device 12 may be one size or may be configured in different sizes for use on different sized patients. The sensor device 12 may be shaped and sized differently for different target areas. The sensor device 12 may be disposable.

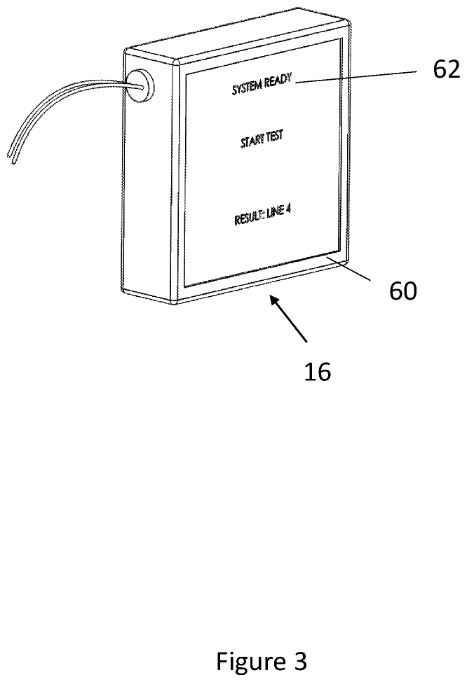

FIG. 3 shows an exemplary computing device 16 according to an aspect of the present invention. The sensors 14 may be coupled to a computing device 16. The computing device 16 may include a screen 60 and a display 62. The computing device 16 may include a keyboard (not shown). Computing device 16 may include software (not shown) for processing the sensed data and calculating an optimal entry point. The computing device 16 may power the sensors 14. In some embodiments, when the program of the computing device 16 is started, the connection with the sensor device 12 may power the sensor device for starting the sensing by the sensors 14. In some embodiments, wherein the computing device 16 does not power the sensors 14, the sensors may be independently powered and after the device 12 is applied to a patient the sensor may start sensing the defined parameter.

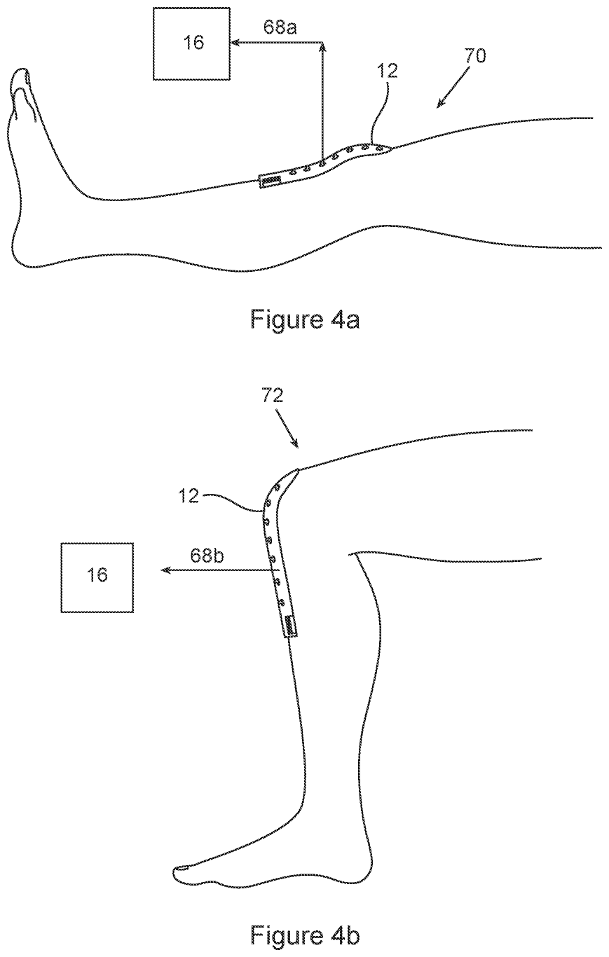

FIGS. 4a and 4b show respectively an exemplary sensor device 12 coupled to the knee in an extended configuration 70 and a bent configuration 72. The computing device 16 may be configured for receiving sensed readings 68 from the sensors 14. The computing device 16 may be configured for analyzing the data 68 received from the sensors. The computing device 16 may build a data file. The computing device 12 may be configured for calculating values relating to the sensed data 68. Each sensor 14 may communicate with the computing device 16 and may transmit values 68 relating to the property sensed, for example the strain and/or displacement from a target area to which the sensor is coupled. For reliable assessment of a point of entry, data 68 may be collected from at least two different orientation states of a target site, such as a first configuration with the target site extended 70 (FIG. 4a) and a second configuration with the target site bent 72 (FIG. 4b). The strain and/or displacement on the bone will not change from a fully extended state to a flexed state of a target site. However, the strain and/or displacement on a body part which is not bone, such as the tendon will change from a fully extended state to a flexed state. The patient may bend or extend a target site to provide the different configurations. The bending of a joint may be about a ninety degrees bend. The bending of a joint may be between about sixty to about ninety degrees. In some embodiments, the patient may bend the target site using any suitable degree of bending. The computing device 16 may receive the two sets of data 68a, 68b from each sensor 14 of the patch 12 in each different extended 70 and flexed state 72 of the target site. A point of entry is optimally a position in the bone where there is no or minimal strain or displacement. Computing device 16 may display the readings 68 from the sensors 14 graphically. The readings 68 from a sensor 14 on the bone may result in a flat line graph, whereas the readings 68 from a sensor 14 on a tendon or other location which is not bone may result in a parabolic graph. A flat or essentially flat line graph may be indicative that the sensor 14 is on an optimal point of entry. In some cases, the sensors 14 may sense a base reading of strain or displacement resulting for example from hair and/or adhesive from the patch. Computing device 16 may employ an algorithm to calculate the difference in the value of the strain or displacement at the position of a sensor, resulting from two different states of flex of a target area. The algorithm may use data resulting from the first configuration and the second configuration, which removes the error from any base reading. In some embodiments, a sensed value from the first configuration may be subtracted from the sensed value from the second configuration and the result recorded. A result of zero or the minimal recorded result may indicate that the sensor 14 is positioned at a potential radial or horizontal line of a suitable access point, and the algorithm may now calculate the most optimal point of entry.

The computing device 16 may be any suitable device which can communicate with the sensors 14 and calculate the point with a difference of zero strain or displacement. Suitable computing devices 16 may include at least one of a smart phone, a smart watch, a computer, a microcomputer and a tablet. The computing device 16 may be coupled to the device 12 featuring the plurality of sensors by a wired connection or wirelessly. In one non-limiting example the computing device 16 may be fixedly attached to the sensor device 12. In such an example the computing device 16 may be disposable.

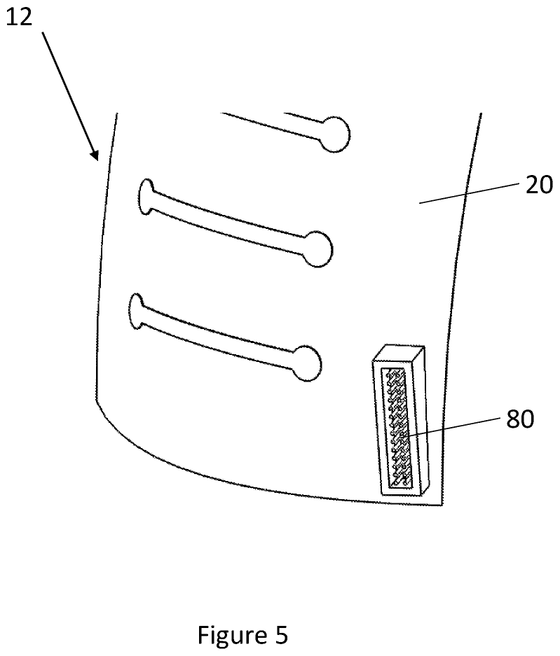

FIG. 5 shows a perspective view of an exemplary electrical connection of an exemplary sensor device. In an embodiment wherein the computing device 16 is connected to the sensor device 12 using a wired connection, the sensor device may include a connection component 80. The connection component 80 may be configured for electrical connection of the computing device 16 to the sensor device 12. The connection component 80 may be any suitable electrical socket. Each of the plurality of sensors 14 may be electrically coupled to the connection component 80 for facilitating connection of the sensors to the computing device 16 when the computing device 16 is coupled to the connection component 80. The connection component 80 may be positioned on any suitable part of the sensor device 12. In one non-limiting example as shown in FIG. 5, the electrical connection component 80 may be disposed on a non-skin facing side 20 of the sensor device 12 for facile connection to an externally located computing device 16 as shown in FIGS. 6a and 6b. FIG. 6a shows computing device 16 coupled to a sensor device 12 with a wired connection featuring wire 24. FIG. 6b shows the wired connected system featuring the sensor device 12 and the computing device 16 attached to the knee of a patient. The computing device 16 may provide the sensors 14 with sufficient power to sense and communicate the data to the computing device 16.

In an embodiment wherein computing device 16 uses a wireless connection to communicate with the sensor device 12, the sensor device may include an antenna 82 and a battery 84 or other suitable power source as shown in FIG. 7. The sensors 14 and identification elements 90 may be connected to the antenna 82 and the battery 84 by for example a wired connection. The antenna 82 may facilitate communication with the computing device 16 and the battery 84 or other power source may provide the sensor device 12 with power to sense and communicate data to the computing device 16. The communication protocol between antenna 82 and the computing device 16 is configured to differentiate between the sensors and the data from each sensor is delivered separately to the computing device 16.

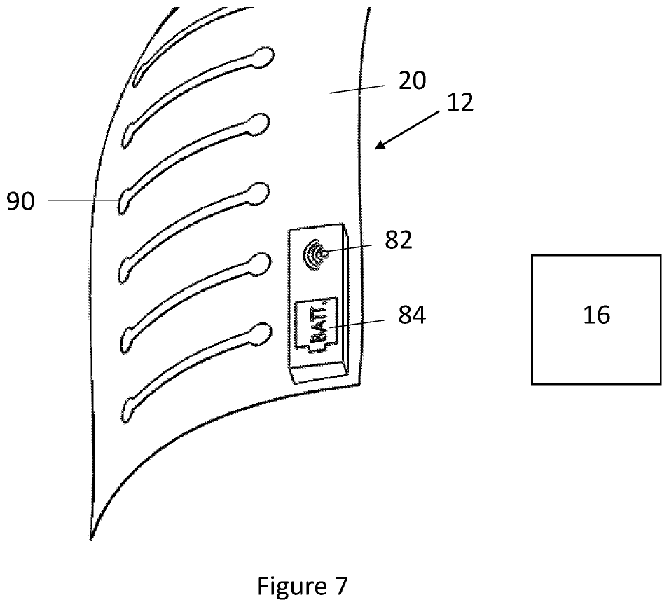

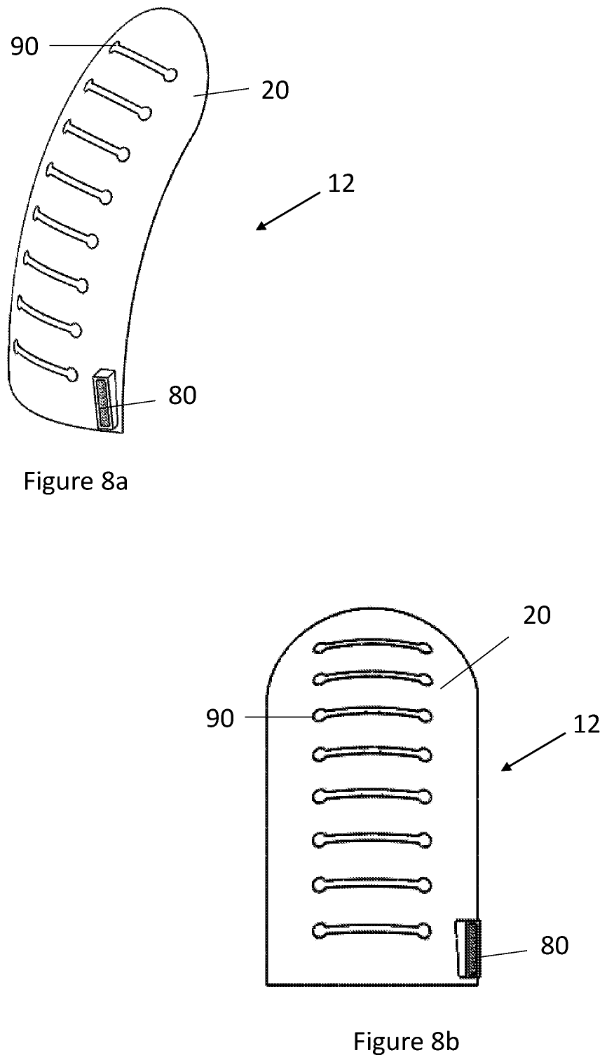

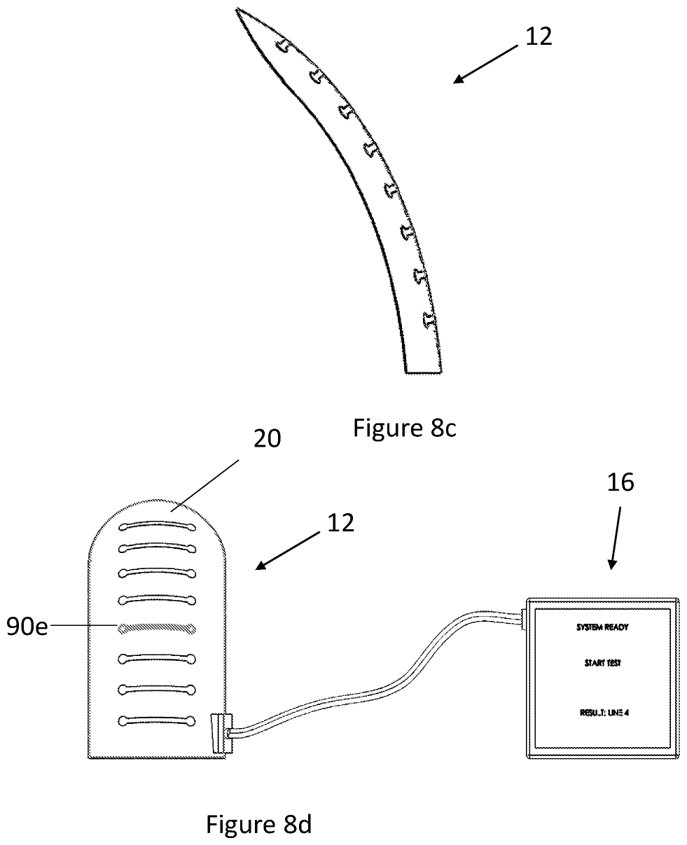

The computing device 16 may relay to a user which sensor 16 is located at an optimal point of entry. The sensor device 12 may include at least one entry point identification or identification element 90. The at least one entry point identification element 90 may be positioned on the non-skin facing side 20 of the sensor device 12 so that it can be viewed by a user. The at least one entry point identification element 90 may be configured to alert a user as to which sensor 14 is determined by the computing device 16 to be located on a zero strain or zero displacement site or minimal calculated displacement site of the target body area. In one embodiment as shown in FIGS. 8a, 8b and 8c, which illustrate different views of the non-skin facing side 20 of the sensor device 12, the sensor device may include at least one entry point identification element 90 featuring a plurality of light emitting diodes (leds) or other suitable lights which can be illuminated to show the user the position of the calculated point of entry. As used herein the term “entry point identification element” is interchangeable with the term “led”. Each of the plurality of leds 90 may be aligned on the non-skin facing side 20 at a position corresponding to a sensor 14 on the skin facing side 18 of the sensor device 12. FIGS. 8a, 8b and 8c show respectively a left side view, a front view and a right-side view of the external facing face 20 of an exemplary sensor device 12 featuring a plurality of leds 90 according to an aspect of the present invention. FIG. 8d shows an exemplary user entry point identification element 90 wherein the led corresponding to the sensor 14 positioned at an optimal entry point is lit up by the computing device 16, which in the example shown in the figure is led 90e.

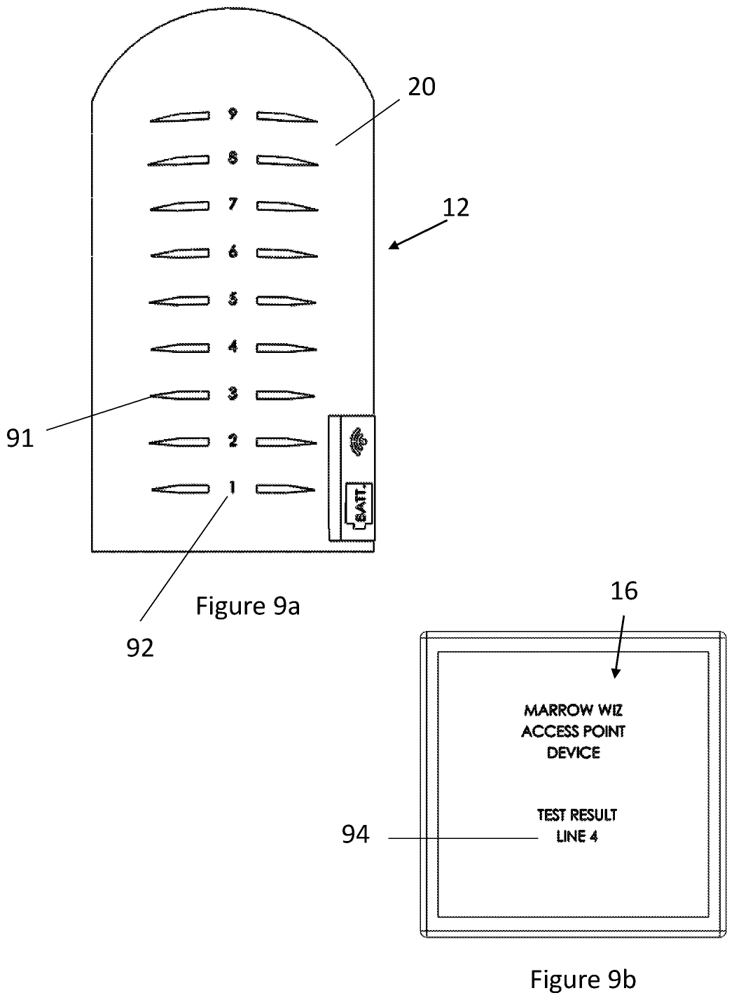

In one embodiment, instead of, or in addition to leds 90 the entry point identification elements 90 may feature markings 91 on the external facing side 20 of the sensor device 12 as shown in FIG. 9a. The markings 91 may correspond to the position of each sensor 14 on the skin facing side 14 of the sensor device 12. The markings 91 may include an identification which can be a number 92 indicating of a sensor to which the position of the marking corresponds, such as but not limited to the identification number of a sensor 14. The computing device 16 may determine which sensor 14 is located at an optimal point of entry and may display this data 94 on the computing device 16. The data 94 may include a sensor identification number, which can be identified by the same number 92 on the external face 20 of the sensor device 12.

In one embodiment, the entry point identification elements 90 may be provided by markings 91 from a laser. The laser markings 91 may be on the skin at the target site and may be employed by the user to accurately locate an access point, such as for a needle and/or a drill at an optimal point of entry at a target site of the body.

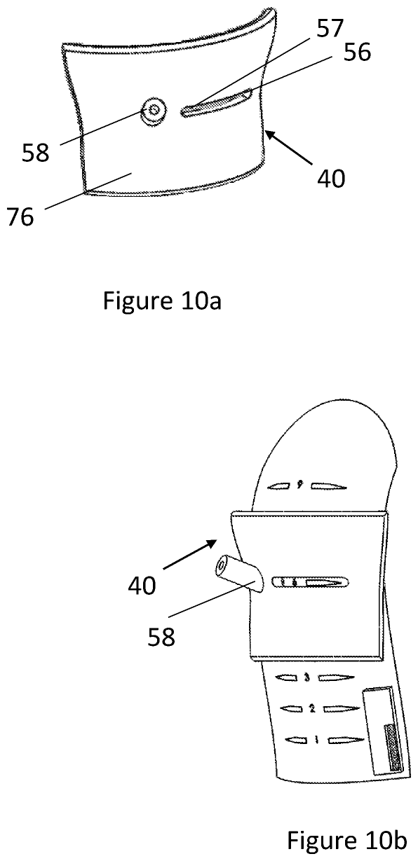

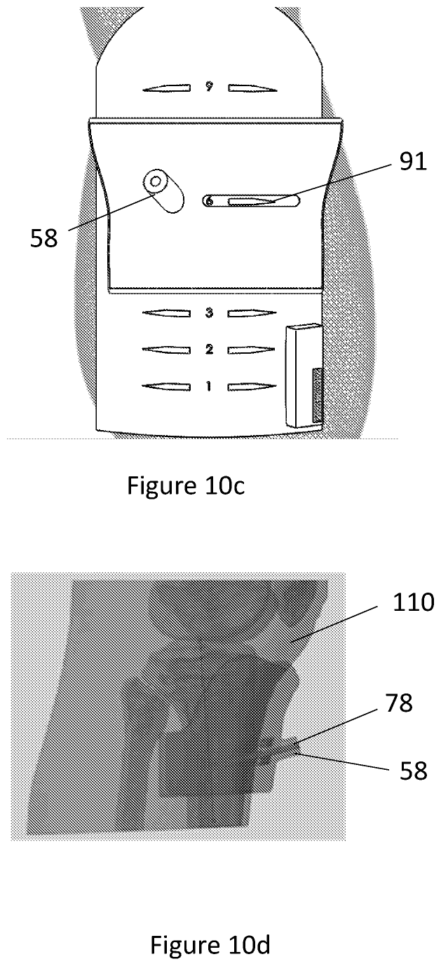

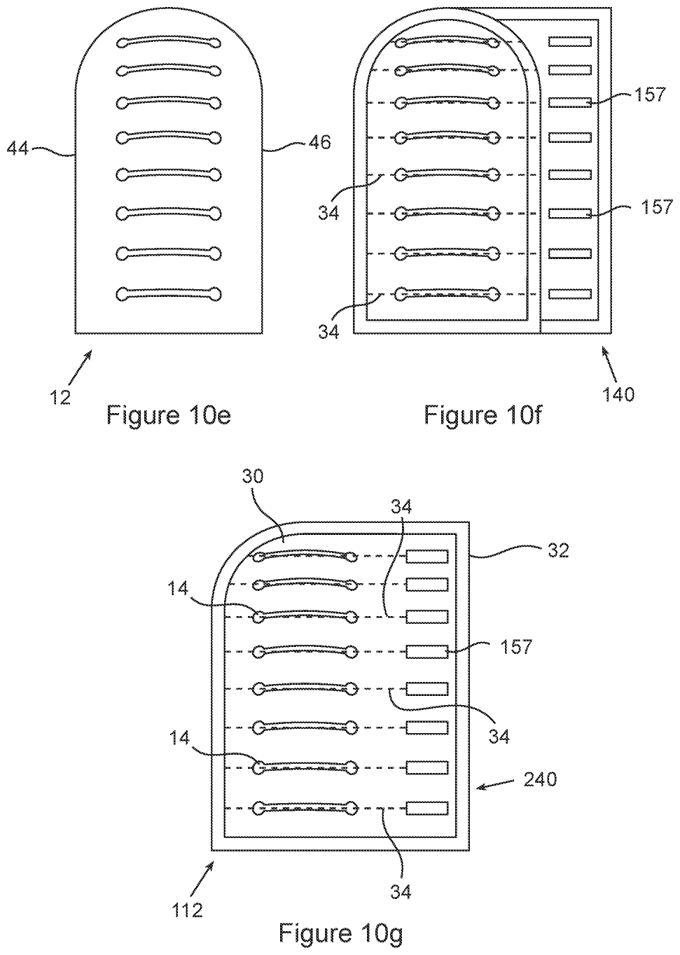

The entry point identification system may include a guiding tool 40 to guide a user to an access point corresponding to the optimal point of entry as determined by the computing device 16 as shown in FIGS. 10a-10g. The access point may be for insertion therein of an instrument, such as but not limited to a needle or a drill or a bone marrow aspiration device.

FIG. 10a shows an access point guiding tool 40. The tool 40 may be attachable to the non-skin facing side 20 of the sensor patch 12 using any suitable attachment means, such as but not limited to at least one strap (not shown). When the user is alerted to which sensor is at an optimal point of entry, the guiding tool 40 may be attached to the non-skin facing side 20 of the senser device 12 and positioned to overlie the indicated sensor 14. The guiding tool 40 may feature a base substrate 76. The base substrate 76 may be made from any suitable material, such as PLA (polylactic acid), TPU (thermoplastic polyurethane) or other non-toxic polymeric material. Coupled to the base substrate 76 may be an alignment means 56 for aligning the guiding tool 40 with the sensor 14 determined to be positioned at an entry point. The alignment means 56 may feature an opening 57 configured to be positioned on the external side of the sensor device 12 so that it overlies a marked entry point identification element 90 of a sensor 14 on the sensor patch 12. The marked entry point identification element 90 may be marked by for example an indicator or a sensor number identification 92. In an embodiment wherein the entry point at the target area has been marked on the skin in any suitable way to indicate the position of the sensor 14 at an optimal entry point, the patch 12 may be removed and the guiding tool 40 may be coupled directed to the skin so that the alignment means 56 overlies the marking on the skin (not shown). FIG. 10b shows the guiding tool 40 attached to the sensor device/patch 12, so that it is aligned with sensor number 6. The guiding tool 40 may include an access hole 58 such as for example a port to receive an instrument such as a needle, the access hole 58 located spaced apart and in the same horizontal line as the alignment means 56. The access hole 58 may be configured for providing a user with an entry point at access hole 58 for access by an instrument to an internal target site. The access hole 58 may be configured to provide the correct trajectory for a needle or a bone aspiration device inserted in the access hole 58. When the guiding tool 40 is secured to the patch 12 on the target area of the body as shown in FIG. 10c, the access hole 58 is configured to provide a trajectory to the target bone for a drill and needle inserted through the access hole 58. A needle 78 may be inserted through the access hole 58 and into the bone 110 as shown in FIG. 10d.

In an alternative embodiment as shown in FIGS. 10e and 10f, a guiding tool 140 may be configured such as a template featuring a plurality of openings 157. The guiding tool 140 may be sized to reversibly attach to the sensor device 12 and provide the plurality of openings 157 at a position adjacent to at least one of the longitudinal extremities 44, 46 of the sensor device 12 wherein the openings 157 are in the same horizontal line 34 as the plurality of sensors 14.

In another exemplary embodiment, a patch 112 may include an integrated guiding tool 240. In such an embodiment, patch 112 may include at least one section 32 with a plurality of openings 157. The patch 112 may be sized so that when applied to a target body area, covering part of the target area is the at least one section 32 of the patch 112. The section 32 spaced apart from the plurality of sensors and extending along the same radial lines 34 as the plurality of sensors 14, such as shown in FIG. 10g. The plurality of openings 157 may be windows, which provide the user with access to the skin for needle penetration. Each opening 157 may be positioned on the same horizonal line 34 as a sensor 14 from the plurality of sensors. The at least one section 32 may extend sideways from section 30 of the patch 112 with the sensors 14. The at least one section 32 may be for providing the user with a point of entry location corresponding to the calculated point of entry for administering an instrument therethrough, such as for example a needle.

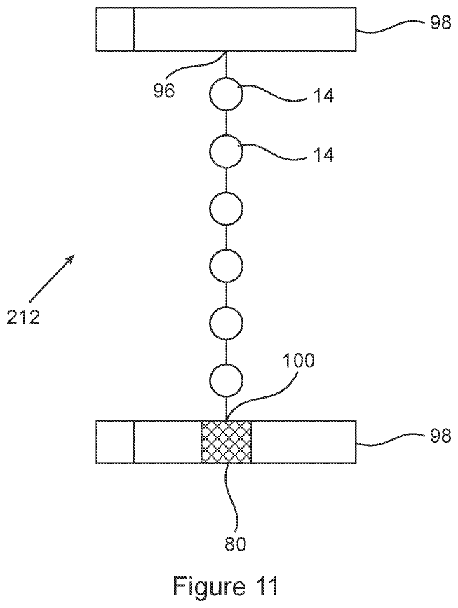

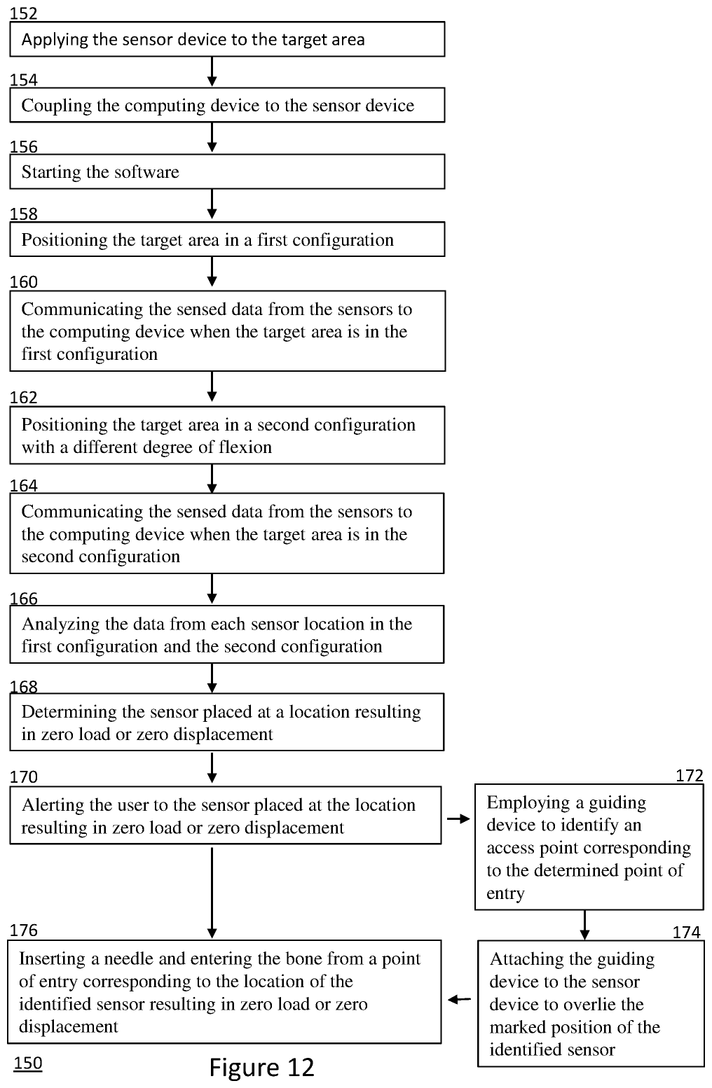

An alternative embodiment of a sensor device 212 is shown in FIG. 11. FIG. 11 shows a sensor device 212, which may feature a plurality of sensors 14 attached to each other to provide a longitudinally spaced apart array of sensors 14 without a base substrate. The proximal end 96 of the array of sensors 14 may include an attachment means 98 for attaching the sensors 14 to a target body area, such as, for example a knee. The attachment means 98 may be for example at least one of a cuff, a bracelet and a band. The distal end 100 of the array of sensors 14 may include a similar attachment means 98 for secure attachment of the array of sensors to a lower region of the target body area. The sensor device 212 and its attachment to a target body area is configured for providing the sensors 14 in contact with the skin of the target area. In an embodiment wherein the computing device 16 uses a wired connection, the sensor device 212 may include a connection component 80 configured for electrical connection of the computing device to the sensor device. The connection component 80 may be positioned on any suitable part of the sensor device 212, such as, but not limited to affixed to the target body site attachment means 98. In an embodiment which employs a wireless connection between the sensor device 212 and the computing device 16, an antenna 82 and a battery 84 may be suitably attached to the sensor device 212. In one non limiting example the antenna 82 and battery 84 may be coupled to the target body area attachment means 98.

The present invention provides a method of use 150 of the entry point identification system. The system may be used in an invasive procedure. One non-limiting example of a suitable invasive procedure is bone marrow aspiration, such as in the patella. A user such as a medical professional may attach a sensor device to the target area, such as knee 152. In an example wherein the sensor device is an adhesive patch, a protective cover may be removed before application when the patch includes such a cover. The patch may be applied to the skin about the knee. The patch may be applied so that the spaced apart sensors are disposed longitudinally on the midline from the patella to the tibial tuberosity along the anterior crest of the tibia. A computing device may be coupled to the sensor device 154. Coupling may be done by connecting the computing device to a corresponding connection component on the sensor device. The user conducting the procedure may start the collecting of data by for example opening and starting a program on the computing device, the program directed to calculating the entry point 156. In a wireless system, coupling the sensor device to the computing device may be done by starting the program on the computing device. The sensors may continually sense a parameter, the parameter depending on the type of sensor being used. The sensor may sense the strain and/or displacement on the target area to which it is coupled. The patient may be instructed to initially place the target area in a first position 158. For example, initially the patient may place the area in a fully extended state. The sensed data may be transmitted from the sensor to computing device 160. After a sufficient time period the patient may be instructed to adopt a different conformation with a different degree of bending 162. The patient may bend the area. In one non-limiting example, the patient may bend the knee ninety degrees. The sensed data resulting from the second configuration of the target body area may be transmitted from the sensor to computing device 164. The computing device may receive both sets of data. The computing device may identify and distinguish between each sensor. The computing device may build a data file. The computing device may analyze the data. The software of the computing device may compare the data from each sensor in the different bending states and may calculate the strain and/or displacement 166. An algorithm may be used to calculate the strain and/or displacement. When the calculated value for a sensor is substantially zero, the computing device may determine that sensor to be at an optimal point of entry 168. The computing device may communicate to the user which sensor is at an optimal point of entry 170. The alerting signal may be the computing device sending a signal to illuminate a led on the external facing side of the sensor device at the position of the identified sensor. The alerting signal may be the computing device displaying the number of the identified sensor and a user identifying the position of the sensor by a marking on the external facing side of the sensor device of for example the sensor identification number which corresponds to the position of the sensor on the skin facing side of the sensor device. The user may employ a guiding device to identify an access point corresponding to the determined point of entry 172. The guiding device may be a device as described hereinabove such as for example the device illustrated in FIGS. 10a-10f. In such an embodiment the guiding device may be suitably attached to the sensor device to overlie the marked position of the sensor corresponding to zero load or zero displacement 174. When suitably attached the access point may be in the correct position for inserting an instrument, such as a needle therethrough. The user may then insert the needle at a suitable position, such as through the access point of the guiding device 176. In an alternative embodiment, the guiding device employed may not be as described in FIGS. 10a-10f, but may be a laser pointing guiding device, which may be used to indicate to the user the access point on the skin of a patient. The sensor device may be for one time use and may be disposable. The order of the steps is not meant to be limiting and any suitable order may be used.

Reference is made to the following example, which together with the above descriptions illustrates the invention in a non-limiting fashion.

### Example 1

A patient undergoes a bone marrow aspiration procedure. Before the procedure, the doctor wants to determine the best point of entry. The doctor applies a sensor patch of the present invention on the knee of the patient. The patient extends his leg so that the knee is not bent. The doctor connects a computing device to the patch and starts the program. The sensors sense the load on the area they are in contact with and transmit this data to the computing device. After several minutes the patient is asked to bend his leg. The sensors sense the load on the same area they are in contact with in this different conformation and the data is transmitted to the computing device. The computing device uses both sets of data points and calculates the strain. The computing device identifies the sensor for which the difference in the load is zero and determines that this sensor is positioned at an optimal point of entry. The computing device lights a led at the identified sensor position on the external face of the patch. The doctor attaches a guiding tool so that it is properly aligned with the identified sensor position. The doctor ensures that the alignment window is correctly positioned over the sensor. The doctor inserts the needle in an adjacent access point on the guiding tool, providing an optimal point of entry to the bone. The patch is discarded.

One skilled in the art can appreciate from the foregoing description that the broad systems, devices, and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

1.-35. (canceled)

36. An entry point location system comprising:
a device comprising a plurality of spaced apart sensors for external reversible attachment to a patient at a target site, wherein the sensors are configured to transmit sensed data; and
a computing device;
wherein the sensors are in communication with the computing device and the computing device is configured to receive the data from the sensors and use the data to calculate the location of an entry point.

37. The location system of claim 36, wherein each of the plurality of sensors comprises a load cell for measuring load or displacement, and/or a bridge resistor for measuring strain.

38. The location system of claim 36, wherein the plurality of spaced apart sensors are disposed longitudinally on the skin facing side of a patch, and, wherein the patch comprises an adhesive on the skin facing side of the patch for detachable attachment to the skin of a patient.

39. The location system of claim 38, wherein the patch is sized and shaped to be placed on the knee, such that the plurality of spaced apart sensors are disposed between about the patella to below the tibial tuberosity.

40. The location system of claim 36, wherein the computing device is at least one of a computer, a microcomputer, a smartphone, a tablet and a smartwatch.

41. The location system of claim 36, wherein the plurality of sensors are coupled by wires to the computing device, or wherein the plurality of sensors are coupled wirelessly to the computing device.

42. The location system of claim 41, wherein the sensor device comprises an attachment component for coupling to the computing device for facilitating communication from the plurality of sensors to the computing device, optionally wherein the computing device powers the plurality of spaced apart sensors.

43. The location system of claim 36, wherein each sensor of the plurality of sensors comprises an identification means corresponding to the sensor to facilitate a user identifying the sensor, wherein the identification means are optionally disposed on the external facing side of the sensor device.

44. The location system of claim 43, wherein the identification means is a led, optionally an identification number.

45. The location system of claim 36, wherein the system is configured to collect data from the plurality of sensors when the target site is flexed and/or when the target site is extended.

46. The location system of claim 36, further comprising a guiding tool for guiding a user to an entry point, wherein the guiding tool comprises a section of the sensor device, the section spaced apart from the plurality of sensors, the section comprising one or more openings positioned on the horizontal lines of each of the plurality of sensors, for providing an opening for needle insertion at the determined entry point.

47. The location system of claim 46, wherein the guiding tool comprises:
a base substrate, the base substrate comprising:
an alignment means for aligning the tool with the sensor determined to be positioned at an entry point;
an access hole spaced apart and in the same horizontal line as the alignment means, the access hole configured for providing access to an instrument to an internal target site; and
attachment means for attaching the guiding tool to the sensor device.

48. The location system of claim 47, wherein the alignment means comprises an opening configured to overlie a marked position of a sensor on the external side of the sensor device.

49. The location system of claim 36, wherein the target site is a knee and wherein the sensor device is configured to attach to the knee for attachment of the plurality of sensors on the midline between the patella and the tibial tuberosity along the anterior crest of the tibia and wherein the sensor device comprises a section spaced apart from the plurality of sensors, the section comprising a plurality of openings positioned on the horizontal lines of each of the plurality of sensors, for providing an opening for needle insertion at the determined entry point.

50. The location system of claim 36, wherein the entry point corresponds to a point on the radial line defined by a sensor which senses a strain or a load and wherein the computing device calculates a difference between two bending states of the target area at the sensor of zero.

51. A method of determining the location of an entry point, the method comprising:
attaching the device of claim 36 to a target area of a patient;
sensing data by the at least one sensors;
transmitting the data to the computing device;
calculating the strain;
indicating to the user the sensor for which a difference in the strain in two bending configurations of a target area, is measured by the computing device as approximating to zero; and
determining the entry point about the sensor corresponding to the difference in strain of zero.

52. The method of claim 51, comprising extending the target area of the patient before the sensing data and flexing the target area about ninety degrees before the sensing data.

53. The method of claim 51, wherein the target area is the knee.

54. The method of claim 51, wherein the determining the entry point comprises finding an entry point on the radial line defined at the position of the sensor indicated to the user, wherein the finding an entry point on the radial line comprises attaching the guiding tool of claim 16, so that the opening of the alignment means overlies the marked position on the external side of the sensor device of the sensor corresponding to a difference in strain of zero to provide the user with an entry point at the spaced apart access hole.

55. The method of claim 51, wherein the sensor device comprises a section spaced apart from the plurality of sensors, the section comprising a plurality of openings positioned on the horizontal lines of each of the plurality of sensors, for providing an opening for needle insertion at the determined entry point and wherein finding an entry point comprises finding the opening corresponding to the sensor indicated to the user for insertion of a needle in the opening.
